(19) United States
(12) Patent Application Publication
(10) Pub. No.: US 2019/0336264 A1
(43) Pub. Date: Nov. 7, 2019

(54) PERCUTANEOUSLY-DEPLOYABLE INTRAVASCULAR EMBOLIC PROTECTION DEVICES AND METHODS

(71) Applicant: Mayo Foundation for Medical Education and Research, Rochester, MN (US)

(72) Inventors: Atta Behfar, Rochester, MN (US); David L. Joyce, Rochester, MN (US); Mandeep Singh, LaCrosse, WI (US); Andre Terzic, Rochester, MN (US)

(73) Assignee: Mayo Foundation for Medical Education and Research, Rochester, MN (US)

(21) Appl. No.: 16/465,336

(22) PCT Filed: Dec. 1, 2017
(86) PCT No.: PCT/US2017/064151

§ 371 Date: May 30, 2019

Related U.S. Application Data
(60) Provisional application No. 62/428,820, filed on Dec. 1, 2016.

(51) Int. Cl. A61F 2/01

(52) U.S. Cl. CPC A61F 2/01; A61F 2002/011; A61F 2230/0069; A61F 2230/0067; A61F 2002/016

(57) ABSTRACT

Embolic protection devices can be used to enhance the treatment of heart conditions such as, but not limited to, heart failure and aortic valve stenosis. For example, this document describes percutaneously-deployable intravascular embolic protection devices and methods for their use. The embolic protection devices can be used to capture and remove embolic materials that could otherwise cause adverse patient effects.

### CROSS-REFERENCE TO RELATED APPLICATIONS

This application claims priority to U.S. Application Ser. No. 62/428,820, filed on Dec. 1, 2016. This disclosure of the prior application is considered part of (and is incorporated by reference in) the disclosure of this application.

### BACKGROUND

### 1. Technical Field

This document relates to devices and methods for the treatment of heart conditions. For example, this document relates to a percutaneously-deployable intravascular embolic protection device.

### 2. Background Information

Heart Failure (HF) affects 5.8 million Americans, with an expanding prevalence as over 670,000 new cases are diagnosed each year. It accounts for $40 billion in health care spending and represents the top Medicare diagnosis-related group for hospital billing. Survival at five years is only 50% from the time of initial diagnosis.

Mechanical Circulatory Support (MCS) been shown to dramatically improve survival and quality of life among patients with end-stage heart failure. Unlike cardiac transplantation, which is limited by a donor pool of around 2,000 organs with over 4,000 patients on a waiting list that increases annually, MCS has the potential to offer treatment to an expanding population of recipients. Based on national inpatient data, an estimated 150,000 patients are currently managed medically despite qualifying for MCS, making it one of the most underutilized treatment options with around 3,000 implants performed annually. Left ventricular assist devices (LVADs) are one type of MCS system.

In addition to heart failure, valvular heart disease is an increasingly more common problem worldwide. With the increase in average age globally and the inadequacy of rheumatic heart disease management, it is anticipated that the number of valvular procedures across the globe will breach 800,000 by 2050. The most pervasive of these procedures is the replacement of the aortic valve. Recently, the use of transaortic valvular implants (TAVI or TAVR) is increasingly utilized both in the United States and Europe, comprising of over 50% of all aortic valve procedures done. The incidence of stroke associated with this procedure is greater than 3%. As such, an easy to use device engineered to integrate with current TAVI systems would dramatically influence the safety of this expanding procedural platform.

### SUMMARY

This document describes devices and methods for the treatment of heart conditions such as, but not limited to, heart failure and aortic valve stenosis. For example, this document describes percutaneously-deployable intravascular embolic protection devices and methods for their use.

While the inventive concepts provided herein are primarily described in the context of TAVI and LVAD, other applications of the concepts are also envisioned and within the scope of this disclosure. For example, the inventive concepts can be applied in the context of other heart valves such as, but not limited to, a prosthetic mitral valve or tricuspid valve. Further, in another implementation the inventive concepts provided herein can be applied in the context of patent foramen ovale PFO closure devices, other septal closure devices, left atrial appendage (LAA) closure devices, and the like. Advantageously, devices described herein provide for lumen patency during use, improving the safety and efficacy of any procedure by preserving blood flow.

In one aspect, this disclosure is directed to an embolic protection device that includes: (i) a cylindrical framework comprised of one or more elongate elements, the cylindrical framework being reconfigurable between a low-profile delivery configuration for containment within a delivery sheath and a diametrically-expanded configuration, the cylindrical framework being open at each end and defining an interior space; and (ii) a filter material disposed within the interior space, the filter material having a pore size that allows blood to pass through the filter material while capturing embolic materials within the filter material, the filter material defining an open passage configured for allowing passage of a catheter through the embolic protection device.

Such an embolic protection device may optionally include one or more of the following features. The filter material may be arranged in a frustoconical shape. The open passage may be located at an apex of the frustoconical shape. The filter material may be supported by a plurality of elongate elements extending within the interior space. The embolic protection device may also include a retrieval cord that, when tensioned, diametrically collapses the cylindrical framework. The embolic protection device may also include a seal at the open passage.

In another aspect, this disclosure is directed to a method of implanting a trans-catheter aortic valve in a native aortic valve of a patient. The method includes: (a) navigating a first delivery sheath through the patient to position a distal end portion of the first delivery sheath in an ascending aorta of the patient; (b) deploying an embolic protection device out from the first delivery sheath to engage with the ascending aorta, wherein the embolic protection device reconfigures from a low-profile delivery configuration to an expanded configuration upon emergence from the first delivery sheath, wherein the embolic protection device includes a filter material disposed within an interior space defined by a cylindrical framework of the embolic protection device, and wherein the filter material defines an open passage; (c) while the embolic protection device is engaged with the ascending aorta, navigating a second delivery sheath through the patient and through the open passage to position a distal end portion of the second delivery sheath in the ascending aorta of the patient adjacent the native aortic valve; (d) while the embolic protection device is engaged with the ascending aorta, deploying the trans-catheter aortic valve out from the second delivery sheath to engage with the native aortic valve; and (e) removing the embolic protection device from the patient after the trans-catheter aortic valve is deployed.

Such a method may optionally include one or more of the following features. The filter material may be configured to capture embolic material released by the implanting of the trans-catheter aortic valve. The embolic protection device may self-expand into engagement with the ascending aorta upon emergence from the first delivery sheath. The embolic protection device may be removed by collapsing the embolic protection device from the expanded configuration and positioning the collapsed embolic protection device in a retrieval catheter.

In another aspect, this disclosure is directed to a method of removing thrombus from a left ventricular assist device (LVAD) while the LVAD is implanted and operating within a patient. The method includes: (1) navigating a delivery sheath through the patient to position a distal end portion of the delivery sheath in an outflow conduit of the LVAD; (2) deploying an embolic protection device out from the delivery sheath to engage with the outflow conduit, wherein the embolic protection device reconfigures from a low-profile delivery configuration to an expanded configuration upon emergence from the delivery sheath, wherein the embolic protection device includes a filter material disposed within an interior space defined by a cylindrical framework of the embolic protection device, and wherein the filter material defines an open passage; (3) injecting a thrombolytic agent into a left ventricle of the patient such that the thrombolytic agent flows into the LVAD and causes detachment of thrombus from the LVAD; (4) collecting at least some of the detached thrombus in the filter material; and (5) removing the embolic protection device from the patient while the thrombus is in the filter material.

Such a method of removing thrombus from a LVAD while the LVAD is implanted and operating within a patient may optionally include one or more of the following features. The method may also include: inserting an aspiration device through the open passage; positioning a distal end portion of the aspiration device adjacent the LVAD; and aspirating at least some of the thrombus using the aspiration device. The method may also include increasing an RPM rate of the LVAD and using echocardiographic visualization to confirm closure of an aortic valve of the patient throughout a cardiac cycle. The embolic protection device may self-expand into engagement with the outflow conduit upon emergence from the delivery sheath. The embolic protection device may be removed by collapsing the embolic protection device from the expanded configuration and positioning the collapsed embolic protection device in a retrieval catheter.

Particular embodiments of the subject matter described in this document can be implemented to realize one or more of the following advantages. Lower adverse event profiles are likely using the devices and methods described herein. In some cases, lower rate of LVAD pump exchange are attainable. Using the devices and methods described herein, shorter lengths of hospital stays are anticipated, along with reduced costs related to LVAD adverse events. Moreover, the eligible LVAD population due to mitigation of one of the primary adverse events associated with this technology is anticipated. It is also envisioned that a retrievable emboli protection system as described herein can be conveniently integrated into existing and future TAVI deployment platforms. Therefore, the emboli protection devices described herein will be readily adopted, and increasing interest in offering this new valvular technology to lower risk candidates will increase adoption of TAVI procedures. In some embodiments, heart conditions such as valvular stenosis can be treated using the devices and methods provided herein. Some patients who would be too high risk for a traditional surgical valve replacement procedure can be treated using the prosthetic valve devices, embolic protection devices, and transcatheter heart valve replacement methods provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of a human heart shown in partial cross-section undergoing a catheterization using a delivery sheath used for deploying an embolic protection device in preparation for a TAVI implant procedure in accordance with some embodiments provided herein.

FIG. 2 is a schematic diagram of the human heart of FIG. 1 showing the embolic protection device implanted in the ascending aorta in accordance with some embodiments provided herein.

FIG. 3 illustrates a delivery sheath for a TAVI device passing through the embodiment protection device.

FIG. 4 illustrates a TAVI device implanted within the native aortic valve annulus. The embolic protection device is still in the ascending aorta in a position such that it can capture emboli that may have been generated during the TAVI device deployment procedure.

FIG. 5 illustrates the retrieval of the embolic protection device in accordance with some embodiments.

FIG. 6 illustrates the completion of the TAVI deployment procedure using the embolic protection device.

FIG. 7 is a perspective view of an example embolic protection device in accordance with some embodiments.

FIG. 8 is a perspective view of another example embolic protection device in accordance with some embodiments.

FIG. 9 schematically illustrates a LVAD device implanted in a patient.

FIG. 10 illustrates an embolic protection device being used during a procedure to remove and capture thrombus from the LVAD device.

FIG. 11 is a side view of another example embolic protection device in accordance with some embodiments.

Like reference numbers represent corresponding parts throughout.

### DETAILED DESCRIPTION

This document describes devices and methods for the treatment of heart conditions such as, but not limited to, heart failure and aortic valve stenosis. For example, this document describes percutaneously-deployable intravascular embolic protection devices and methods for their use.

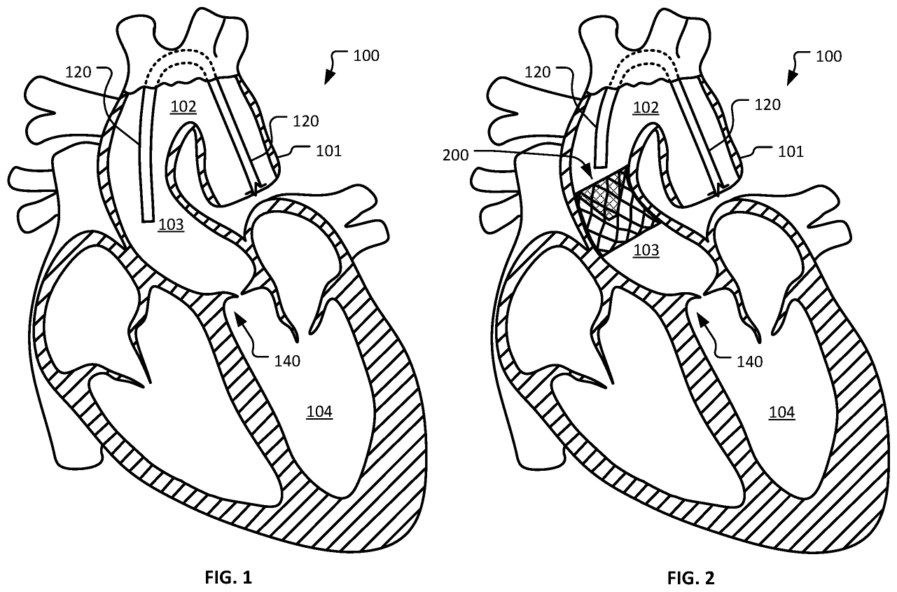

With reference to FIG. 1, a schematic diagram is provided of human heart 100 shown in partial cross-section undergoing a catheterization of aorta 101 using a delivery sheath 120. Delivery sheath 120 is in aorta 101 for the purpose of transmitting an embolic protection device (not visible because the embolic protection device is in a low-profile delivery configuration within delivery sheath 120) to be implanted within ascending aorta 103.

In some cases, delivery sheath 120 can be percutaneously inserted in a femoral artery of a patient, and navigated to the patient's aorta 101 using imaging techniques such as fluoroscopy, MRI, or ultrasound. In some circumstances, a guidewire may be installed first. Radiopaque and/or echogenic markers can be included on delivery sheath 120 for enhanced imaging. Within aorta 101, delivery sheath 120 can be directed to aortic arch 102 and then to ascending aorta 103 towards native aortic valve 140. In other cases, aorta 101 can be accessed by delivery sheath 120 via the patient's radial artery. Other aortic access techniques are also envisioned, such as a transapical approach or a transvenous transeptal approach.

With reference to FIG. 2, an embolic protection device 200 is shown after being deployed from delivery sheath 120 to a diametrically expanded configuration and implanted within ascending aorta 103. The stent portion of embolic protection device 200 is visible. The stent portion is a generally cylindrical framework of elongate elements that conforms to the anatomy of the patient at the implant site. In some embodiments, embolic protection device 200 is self-expanding. That is, embolic protection device 200 can be configured to self-expand after being released from the diametrically-constraining confines of delivery sheath 120. In some embodiments, embolic protection device 200 is balloon expandable. That is, embolic protection device 200 can be configured to expand in response to radially-directed outwardly-expansive forces from the inflation of a balloon disposed within the center of embolic protection device 200.

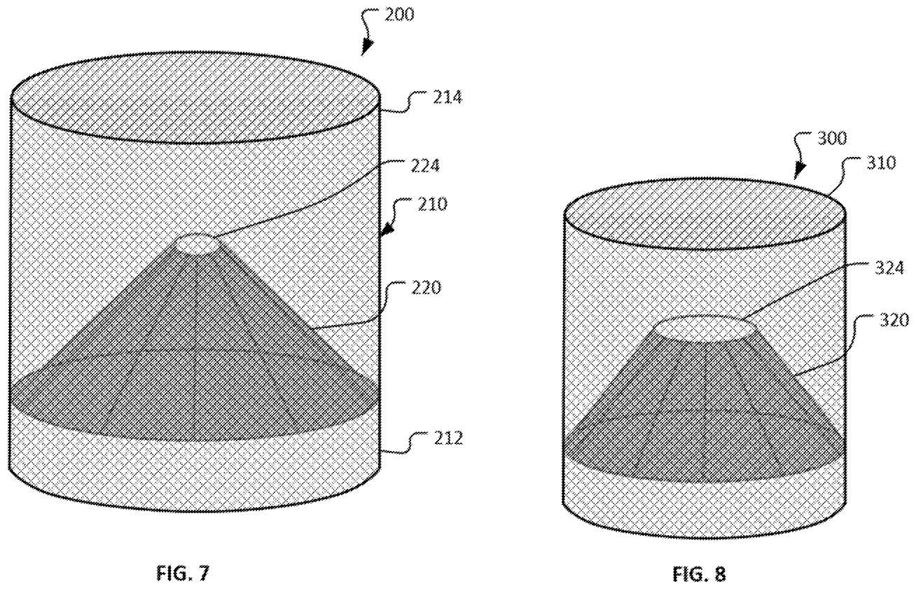

Referring to FIG. 7, embolic protection device 200 is shown in greater detail. Embolic protection device 200 includes an outer stent frame 210, an inner filter 220, and a passage 224. Inner filter 220 is disposed within stent frame 210. Inner filter defines passage 224.

In some embodiments, outer stent frame 210 is a laser-cut, expanded, and heat-set metallic frame. For example, in some embodiments a super-elastic material such as nitinol (NiTi) is used for the material of outer stent frame 210. In some embodiments, stainless steel is used for the material of outer stent frame 210. In some embodiments, outer stent frame 210 is wire-wound, and may comprise one or more wires. Such a construct may be woven, a mesh, braided, and/or the like. In some embodiments, one or more portions of outer stent frame 210 are covered by material (e.g., Dacron, polyester fabrics, polyethylene terephthalate (PET), Teflon-based materials, Polytetrafluoroethylene (PTFE), expanded Polytetrafluoroethylene (ePTFE), polyurethanes, silicone, Bio A, copolymers, film or foil materials, or combinations of the foregoing materials and/or like materials). Such covering materials may provide enhanced sealing and/or blood flow barriers between outer stent frame 210 and the tissues against which it abuts.

In some embodiments, outer stent frame 210 include one or more visualization markers, such as radiopaque or echogenic markers, bands, or radiopaque filler materials. The radiopaque or echogenic markers can assist clinician (such as an interventional cardiologist) with in situ radiographic visualization of embolic protection device 200 so that the clinician can orient the device as desired in relation to the anatomy of the patient.

Outer stent frame 210 includes a proximal end 212 and a distal end 214. The ends 212 and 214 of outer stent frame 210 are open. That is, like a hollow cylinder that defines an interior space, each end 212 and 214 of stent frame 210 is open to receive/convey blood flow. However, blood flowing through embolic protection device 200 must pass through inner filter 220 to travel from one end 212/214 of stent frame 210 to the other end 214/212 of stent frame 210.

Inner filter 220 is coupled to outer stent frame 210 within the periphery of outer stent frame 210. In some embodiments, inner filter 220 is constructed of one or more frame members that extend within the interior space defined by outer stent frame 210 and a filter media/material. Such frame members can provide support and rigidity to otherwise flaccid filter media/material. In some embodiments, the filter media/material is attached to frame members of inner filter 220 by mechanisms such as, but not limited to, suturing, using mechanical clips, sewing, using adhesives, bonding, a mechanical channel, and by combinations thereof.

The filter material of inner filter 220 can be configured with a pore size that allows blood to pass therethrough, while capturing embolic materials such as, but not limited to, thrombus, plaque, tissue particles, and the like. In some embodiments, such as the depicted embodiment, inner filter 220 is configured in a conical or frustoconical shape. Such a conical shape can provide additional filter area in some cases (e.g., as compared to a planar-shaped filter). In some embodiments, inner filter 220 is generally planar.

Inner filter 220 defines a passage 224. As described further below, passage 224 can allow catheters and instruments of various kinds to pass through embolic protection device 200. In some embodiments, a resilient seal is included at passage 224. In some embodiments, passage 224 is located at an apex of the conical or frustoconical shape.

As described further below, embolic protection device 200 is configured to be retrievable. That is, after expression from a delivery sheath, expansion and use, embolic protection device 200 can thereafter be retrieved into a sheath for removal from the vascular system. This retrievability can be accomplished in various ways.

In some embodiments, various portions of embolic protection device 200 include eyelets through which a retrieval cord (i.e., a lasso) is threaded. For example, in some embodiments a single end of stent frame (e.g., proximal end 212 of the stent frame 210) has eyelets. In other embodiments, the opposite end (distal end 214), or both ends 212 and 214 can have eyelets for a retrieval cord. Such eyelets and retrieval cords are used to retrieve or reposition embolic protection device 200. For example, in some cases a grasping device (not shown) can be routed to the site of embolic protection device 200 (such as through the delivery sheath or independently), and the grasping device can be used to attach onto a retrieval cord. The grasping device can be used to pull on retrieval cord, which causes the eyelets to collapse toward each other like a purse when a purse string is used to cinch the purse closed. In the collapsed configuration, embolic protection device 200 can be repositioned or retrieved into a sheath for removal from the patient's body. In some embodiments, such retrieval cord(s) remains coupled to stent frame 210 when embolic protection device 200 is in use in a patient. Retrieval cords can be made of polymer materials such as, but not limited to, nylon, polypropylene, PTFE, silk, and the like. In some embodiments, retrieval cords can be a wire made of a metallic material including, but not limited to, nitinol, aluminum, stainless steel, and the like.

Also referring to FIG. 8, it should be appreciated that the embolic protection devices described herein are scalable to any suitable size in accordance with a variety of desired end uses and patient anatomies. For example, the embolic protection device 300 is smaller in diameter and length than embolic protection device 200, but has a larger passage 324 than passage 224 of embolic protection device 200. Any combination of shapes and sizes are included within the scope of this disclosure.

In some embodiments, such as for the TAVI implant procedure illustrated in FIGS. 1-6, outer stent frame 210/310 has an outer diameter of about 30 mm to about 40 mm, or about 25 mm to about 45 mm, or about 20 mm to about 50 mm. In some embodiments, such as for the TAVI implant procedure illustrated in FIGS. 1-6, passage 224/324 has a diameter of about 3 mm to about 6 mm, or about 2 mm to about 7 mm.

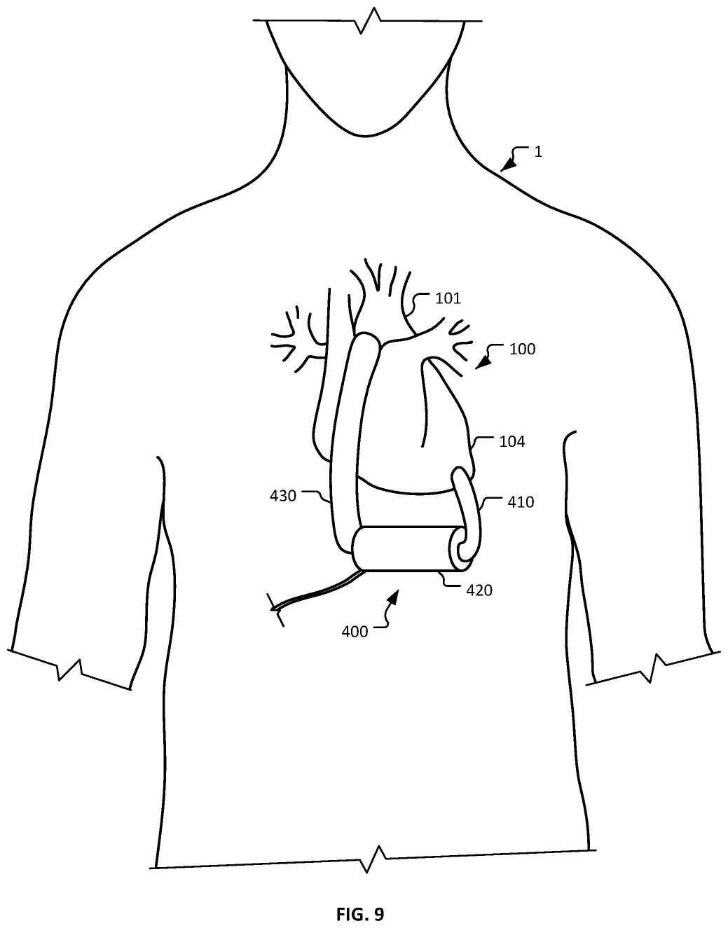

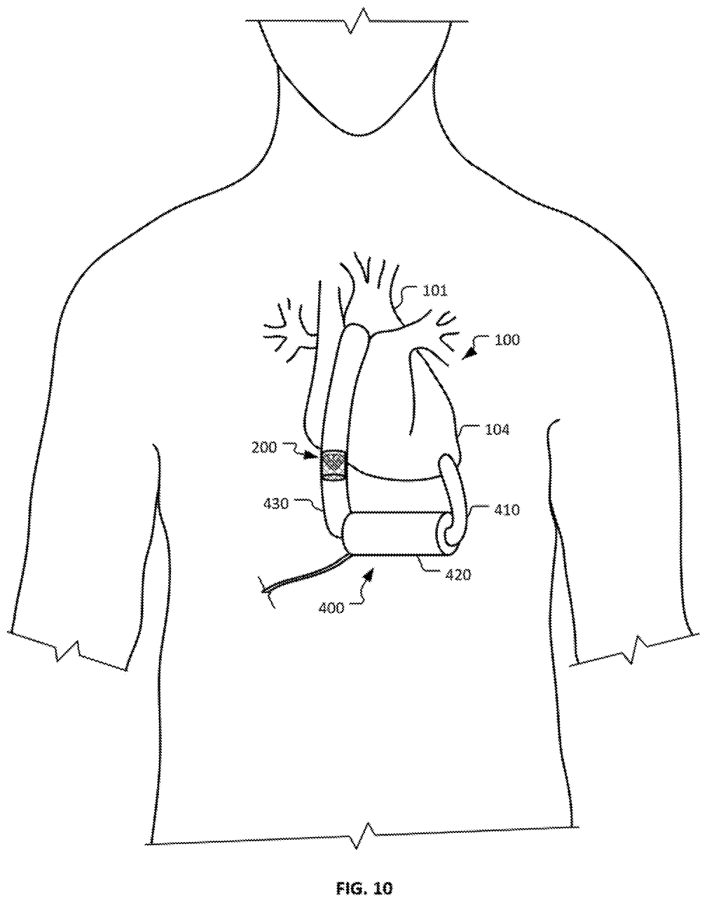

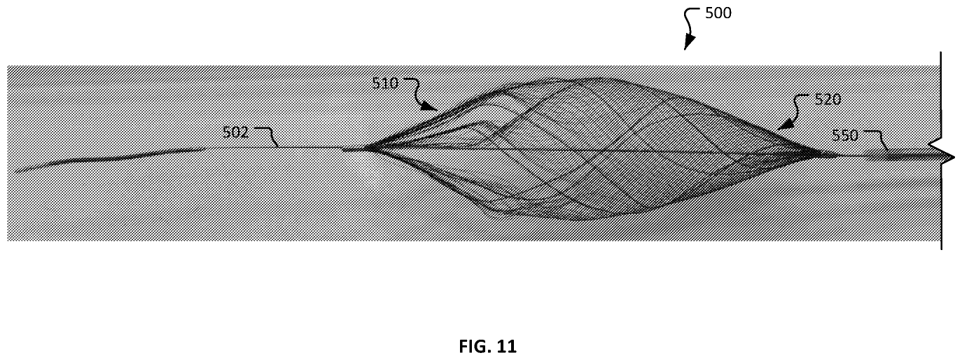

In some embodiments, such as for the LVAD thrombosis removal procedure illustrated in FIGS. 9 and 10, outer stent frame 210/310 has an outer diameter of about 12 mm to about 18 mm, or about 10 mm to about 20 mm, or about 8 mm to about 22 mm. In some embodiments, such as for the LVAD thrombosis removal procedure illustrated in FIGS. 9 and 10, passage 224/324 has a diameter of about 1.5 mm to about 2.5 mm, or about 1 mm to about 3 mm.

Again, it should be understood that the sizes provided above are purely illustrative, and that any size, shape, and combinations of sizes and/or shapes are envisioned within the scope of this disclosure.

Still referring to FIG. 2, embolic protection device 200 is shown situated in ascending aorta 103 after deployment from delivery sheath 120. Blood flowing through aorta 101 passes through embolic protection device 200.

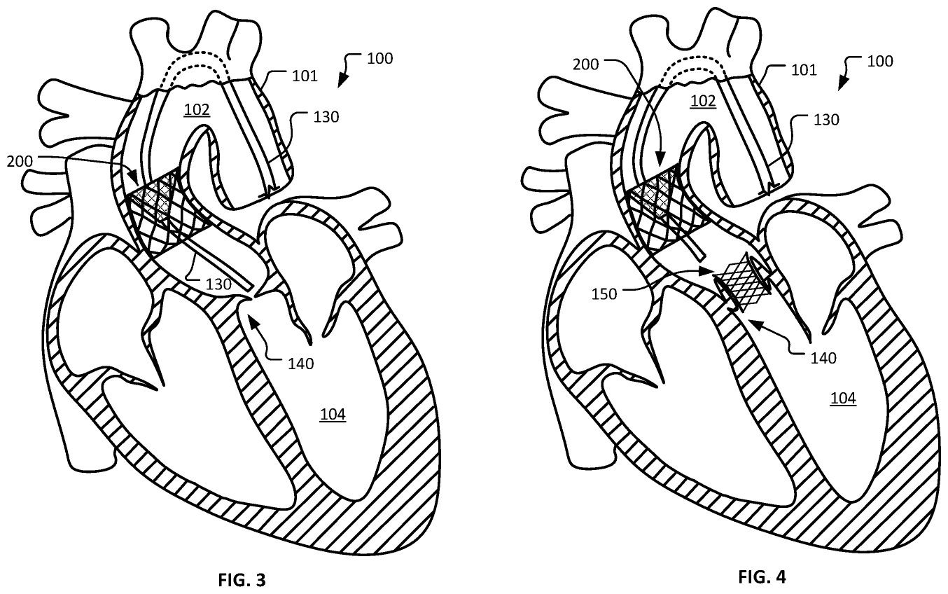

Next, as depicted in FIG. 3, a TAVI delivery catheter 130 is advanced through aorta 101 and through embolic protection device 200 toward native aortic valve 140. In some cases, the delivery catheter used for deploying the TAVI valve can be the same catheter as was used for deploying embolic protection device 200. Delivery catheter 130 can be passed through passage 224 of inner filter 220. During the movements of delivery catheter 130, emboli created/released in the process can be captured by inner filter 220.

Referring to FIG. 4, a TAVI valve 150 can be deployed from delivery catheter 130 and positioned within native aortic valve 140. During the process of implanting TAVI valve 150 in native aortic valve 140, emboli created/released in the process can be captured by inner filter 220. Hence, the risk of stroke related to the TAVI procedure is thereby substantially mitigated.

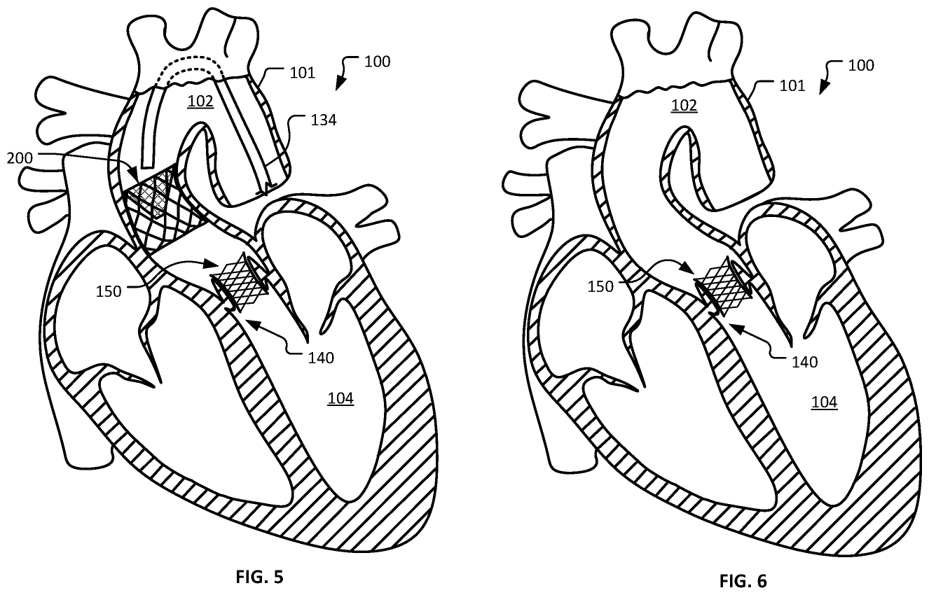

Referring to FIG. 5, after withdrawing delivery catheter 130 from embolic protection device 200, embolic protection device 200 can be retrieved and removed from heart 100. In some cases, embolic protection device 200 can be retrieved into a retrieval sheath 134. In some cases, embolic protection device 200 can be retrieved into either delivery sheath 120 (FIGS. 1 and 2) or TAVI delivery catheter 130 (FIGS. 3 and 4). The retrieval process of embolic protection device 200 is designed to be performed without releasing embolic material(s) that were captured by inner filter 220.

Referring to FIG. 6, after embolic protection device 200 is retrieved and retrieval sheath 134 is removed from aorta 101, prosthetic TAVI valve 150 hereafter takes over the function of the patient's natural aortic valve 140. In the foregoing manner (as described in reference to FIGS. 1-8), the deployment of TAVI valve 150 can be performed while embolic protection device 200 captures embolic materials that release or get generated during the TAVI deployment procedure. Hence, the risk of stroke related to the deployment of TAVI valve 150 can be substantially mitigated using embolic protection device 200.

Referring to FIG. 9, a patient 1 can be treated using an LVAD system 400 that assists the pumping action of patient's heart 100. In some cases, patient 1 may be using LVAD system 400 because of experiencing heart failure.

LVAD system 400 helps the left ventricle 104 pump blood to the patient's body 1. Accordingly, an inflow conduit 410 conveys blood from left ventricle 104 to the inlet of LVAD pump 420. An outflow conduit 430 is coupled to the outlet of LVAD pump 420. Outflow conduit 430 conveys blood that has been pressure-boosted by LVAD pump 420 to aorta 101. From aorta 101, blood flows through the vasculature of patient 1 and returns to heart 100.

One known issue with LVAD systems such as LVAD system 400 is thrombotic complications. Thrombotic complications due to thrombosis formed in LVAD pump 420 can occur. Currently, thrombosis in LVAD systems is commonly treated by replacing the LVAD pump 420 with a new LVAD pump. However, such procedures are highly invasive, requiring cardiopulmonary bypass, and are associated with considerable morbidity and the potential for mortality. Even in an uncomplicated LVAD device exchange, hospitalization is typically required for a week or more. Lytic therapy is another alternative for treating thrombotic complications due to an LVAD. However, with lytic therapy there is a high risk of cerebrovascular accidents (CVA).

Referring to FIG. 10, this disclosure describes a minimally-invasive technique for removing thrombosis from LVAD pump 420. In an effort to mitigate risks associated with typical remedial actions used in response to thrombosis in LVAD pump 420, and to expand the population of heart failure patients that could benefit from MCS therapy, embolic protection device 200 can be utilized as follows.

First, in some embodiments the treatment method involves increasing the RPM rate of LVAD pump 420 under echocardiographic guidance to confirm closure of the aortic valve throughout the cardiac cycle. Embolic protection device 200 can then be deployed in the outflow conduit 430 of LVAD system 400. For example, the deployment can be performed using the techniques described above in reference to FIGS. 1 and 2.

Thrombolytic agents can then be administered, such as into left ventricle 104. The thrombolytic agents will then pass through inflow conduit 410 to LVAD pump 420 where the agent(s) will flush through the impellor and other interior structures of LVAD pump 420. Thrombus in LVAD pump 420 will be dislodged and thereafter flow through a first portion of outflow conduit 430 to be captured within embolic protection device 200. In some embodiments, a thrombus aspiration device (e.g., the AngioJet™ Thrombectomy System from Boston Scientific Corp.) and/or an aspiration catheter of a cell saver system can be deployed through passage 224 of embolic protection device 200 to further enhance thrombus removal. In some embodiments, an aspiration/suction device can enhance thrombus removal and/or thrombolytics removal so that systemic distribution of thrombolytic agents is mitigated.

By limiting drug exposure to LVAD system 400 itself, and by collecting any debris that may be flushed through LVAD system 400, this treatment method using embolic protection device 200 has the potential to minimize complications in a non-invasive fashion. The entire procedure can be accomplished via peripheral arterial access in two locations (e.g., right radial and right femoral). Accordingly, this procedure may be utilized as maintenance therapy to control chronic pump thrombosis in the outpatient setting.

Referring to FIG. 11, another example filter device 500 includes large openings its distal end 510 to allow emboli entry and small openings at its proximal end 520 to trap emboli. Filter device 500 can be used, for example, in conjunction with the procedures described herein. Other uses will also be apparent to those skilled in the art.

Filter device 500 includes a wire 502 to which the mesh filter is coupled. Filter device 500 can self-expand once emerged from a sheath 550, can be pull back into sheath 550 for retrieval after use.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. An embolic protection device comprising:
a cylindrical framework comprised of one or more elongate elements, the cylindrical framework being reconfigurable between a low-profile delivery configuration for containment within a delivery sheath and a diametrically-expanded configuration, the cylindrical framework being open at each end and defining an interior space; and
a filter material disposed within the interior space, the filter material having a pore size that allows blood to pass through the filter material while capturing embolic materials within the filter material, the filter material defining an open passage configured for allowing passage of a catheter through the embolic protection device.

2. The embolic protection device of claim 1, wherein the filter material is arranged in a frustoconical shape.

3. The embolic protection device of claim 2, wherein the open passage is located at an apex of the frustoconical shape.

4. The embolic protection device of claim 1, wherein the filter material is supported by a plurality of elongate elements extending within the interior space.

5. The embolic protection device of claim 1, comprising a retrieval cord that, when tensioned, diametrically collapses the cylindrical framework.

6. The embolic protection device of claim 2, comprising a seal at the open passage.

7. A method of implanting a trans-catheter aortic valve in a native aortic valve of a patient, the method comprising:
navigating a first delivery sheath through the patient to position a distal end portion of the first delivery sheath in an ascending aorta of the patient;
deploying an embolic protection device out from the first delivery sheath to engage with the ascending aorta, wherein the embolic protection device reconfigures from a low-profile delivery configuration to an expanded configuration upon emergence from the first delivery sheath, wherein the embolic protection device includes a filter material disposed within an interior space defined by a cylindrical framework of the embolic protection device, and wherein the filter material defines an open passage;
while the embolic protection device is engaged with the ascending aorta, navigating a second delivery sheath through the patient and through the open passage to position a distal end portion of the second delivery sheath in the ascending aorta of the patient adjacent the native aortic valve;
while the embolic protection device is engaged with the ascending aorta, deploying the trans-catheter aortic valve out from the second delivery sheath to engage with the native aortic valve; and
removing the embolic protection device from the patient after the trans-catheter aortic valve is deployed.

8. The method of claim 7, wherein the filter material is configured to capture embolic material released by the implanting of the trans-catheter aortic valve.

9. The method of claim 7, wherein the embolic protection device self-expands into engagement with the ascending aorta upon emergence from the first delivery sheath.

10. The method of claim 7, wherein the embolic protection device is removed by collapsing the embolic protection device from the expanded configuration and positioning the collapsed embolic protection device in a retrieval catheter.

11. A method of removing thrombus from a left ventricular assist device (LVAD) while the LVAD is implanted and operating within a patient, the method comprising:
navigating a delivery sheath through the patient to position a distal end portion of the delivery sheath in an outflow conduit of the LVAD;
deploying an embolic protection device out from the delivery sheath to engage with the outflow conduit, wherein the embolic protection device reconfigures from a low-profile delivery configuration to an expanded configuration upon emergence from the delivery sheath, wherein the embolic protection device includes a filter material disposed within an interior space defined by a cylindrical framework of the embolic protection device, and wherein the filter material defines an open passage;
injecting a thrombolytic agent into a left ventricle of the patient such that the thrombolytic agent flows into the LVAD and causes detachment of thrombus from the LVAD;
collecting at least some of the detached thrombus in the filter material; and
removing the embolic protection device from the patient while the thrombus is in the filter material.

12. The method of claim 11, further comprising:
inserting an aspiration device through the open passage;
positioning a distal end portion of the aspiration device adjacent the LVAD; and
aspirating at least some of the thrombus using the aspiration device.

13. The method of claim 11, further comprising increasing an RPM rate of the LVAD and using echocardiographic visualization to confirm closure of an aortic valve of the patient throughout a cardiac cycle.

14. The method of claim 11, wherein the embolic protection device self-expands into engagement with the outflow conduit upon emergence from the delivery sheath.

15. The method of claim 11, wherein the embolic protection device is removed by collapsing the embolic protection device from the expanded configuration and positioning the collapsed embolic protection device in a retrieval catheter.
